(19) United States
(12) Patent Application Publication
(10) Pub. No.: US 2021/0031046 A1
(43) Pub. Date: Feb. 4, 2021

(54) BATTERY ASSEMBLY FOR MEDICAL DEVICE

(71) Applicant: Medtronic, Inc., Minneapolis, MN (US)

(72) Inventor: Hailiang Zhao, Plymouth, MN (US)

(21) Appl. No.: 16/530,470

(22) Filed: Aug. 2, 2019

(51) Int. Cl. A61N 1/378; H01M 2/26; A61N 1/39

(52) U.S. Cl. CPC A61N 1/3787; H01M 2/266; H01M 2220/30; A61N 1/3975; A61N 1/39622

(57) ABSTRACT

In some examples, a battery assembly for an implantable medical device includes an electrode stack comprising a plurality of electrode plates. The plurality of electrode plates comprises a first electrode plate including a first tab extending from the first electrode plate and a second electrode plate including a second tab extending from the second electrode plate; a spacer between a first portion of the first tab and a second portion of the second tab, wherein a third portion of the first tab and a fourth portion of the second tab are joined together adjacent to the first portion, second portion, and the spacer; and a penetration weld that extends through the third portion of the first tab and the fourth portion of the second tab.

### TECHNICAL FIELD

The disclosure relates to batteries and, more particularly, to batteries of medical devices.

### BACKGROUND

Medical devices such as implantable medical devices (IMDs) include a variety of devices that deliver therapy (such as electrical simulation or drugs) to a patient, monitor a physiological parameter of a patient, or both. IMDs typically include a number of functional components encased in a housing. The housing is implanted in a body of the patient. For example, the housing may be implanted in a pocket created in a torso of a patient. The housing may include various internal components such as batteries and capacitors to deliver energy for therapy delivered to a patient and/or to power circuitry for monitoring a physiological parameter of a patient and controlling the functionality of the medical device.

### SUMMARY

In some aspects, the disclosure is directed to battery assemblies for use, e.g., in a medical device, and techniques for manufacturing battery assemblies.

In one example, the disclosure is directed to a battery assembly for an implantable medical device, the assembly comprising an electrode stack comprising a plurality of electrode plates, wherein the plurality of electrode plates comprises a first electrode plate including a first tab extending from the first electrode plate and a second electrode plate including a second tab extending from the second electrode plate; a spacer between a first portion of the first tab and a second portion of the second tab, wherein a third portion of the first tab and a fourth portion of the second tab are joined together adjacent to the first portion, second portion, and the spacer; and a penetration weld that extends through the third portion of the first tab and the fourth portion of the second tab.

In another example, the disclosure is directed to a method for forming a battery assembly, the method comprising assembling an electrode stack with a spacer, electrode stack comprising a plurality of electrode plates, wherein the plurality of electrode plates comprises a first electrode plate including a first tab extending from the first electrode plate and a second electrode plate including a second tab extending from the second electrode plate, wherein the spacer is between a first portion of the first tab and a second portion of the second tab when the electrode stack is assembled with the spacer; joining a third portion of the first tab and a fourth portion of the second tab together adjacent to the first portion, second portion, and the spacer; and welding the electrode stack to form a penetration weld that extends through the third portion of the first tab and the fourth portion of the second tab.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram that illustrates an example medical device system that may be used to deliver therapy to a patient.

FIG. 2 is a conceptual diagram illustrating a partial exploded view of the IMD of FIG. 1.

FIGS. 3 and 4 are conceptual diagrams illustrating portions of an example battery assembly in accordance with examples of the disclosure.

FIG. 5 is a conceptual diagram illustrating a portion of an example battery assembly including a stack of tabs and spacers of an electrode.

FIG. 6 is a conceptual diagram illustrating a cross-sectional view of example stacks of tabs and spacers.

FIG. 7 is a conceptual diagram illustrating a partial plan view of the example stack of FIG. 6.

FIG. 8 is a flowchart illustrating an example technique in accordance with examples of the disclosure.

FIG. 9 is a conceptual diagram illustrating an example cross-sectional view of one of the stacks of tabs and spacers of FIG. 6 prior to the tabs being joined together according to the technique of FIG. 8.

### DETAILED DESCRIPTION

A variety of medical devices may utilize one or more batteries as a power source for operational power. For example, an implantable medical device (IMD) that provides cardiac rhythm management therapy to a patient may include a battery to supply power for the generation of electrical therapy or other functions of the IMD. For ease of illustration, examples of the present disclosure will be described primarily with regard to batteries employed in IMDs that provide cardiac rhythm management therapy. However, as will be apparent from the description herein, examples of the disclosure are not limited to IMDs that provide such therapy. For example, in some instances, one or more of the example batteries described herein may be used by a medical device configured to deliver electrical stimulation to a patient in the form of neurostimulation therapy (e.g., spinal cord stimulation therapy, deep brain stimulation therapy, peripheral nerve stimulation therapy, peripheral nerve field stimulation therapy, pelvic floor stimulation therapy, and the like). In some examples, example batteries of this disclosure may be employed in medical devices configured to monitor one or more patient physiological parameters, e.g., by monitoring electrical signals of the patient, alone or in conjunction with the delivery of therapy to the patient.

In some examples, a battery of an IMD may include a plurality of electrode plates (e.g., including both anode and cathode plates) stacked on each other in which each of the plates includes a tab extending therefrom. The tabs of the anode plates may be aligned with each other in a stack and electrically connected to each other to form an anode of the battery. In this sense, the tab stack may function as an electrical interconnect between the plates of the anode. Similarly, the tabs of the cathode plates may be aligned with each other in a stack and electrically connected to each other to form a cathode of the battery. In some examples, such a battery may be refereed to as a flat plate battery.

In some examples, in each of the anode tab stack and cathode tab stack, a spacer may be located between adjacent individual tabs in the stack of tabs, e.g., such that each individual tab is separated from an adjacent tab by a spacer. The spacers may be electrically conductive to electrically couple the respective tabs in the stack to each other and define an interconnect between respective plates of the electrode. For each electrode, the tabs in the stack of tabs and spacers may be attached to each other by one or more side laser welds that span the height of the tab stack.

During assembly, the electrode plates may be stacked using a fixture pin for alignment. Each tab of the plates may include an aperture, e.g., in the center of the tab, that is inserted onto the fixture pin. The tabs of the plates may be sequentially inserted onto the fixture pin along with any spacers between the tabs to stack the plates with the tabs aligned with each other and spaced as desired. Once stacked, the side of the tab stack may be welded to form one or more side welds that attach the tabs and spacers to each other as a stack of electrode plates. The stack of electrode plates may then be removed from the fixture pin and then sealed within a battery housing.

In some examples, the stack of electrode plates may be subject to “fanning” (e.g., opening like the pages of a bound book) or other forces, e.g., as a result of the mechanical force applied by the expansion of the electrode stack during discharge of the battery. In some examples, the applied force may result in a concentration of stress at the root of the side weld(s) attaching the plates and spacers to each other. Such stress may cause the side weld(s) to fail resulting in undesirable electrical connection between the electrodes and leading to reduced battery capacity and power capability. Weld failure may also result in a spacer breaking away from the stack and may cause internal shorting and undesired reduction of battery capacity and power.

In accordance with at least some examples of the disclosure, a battery assembly that includes an electrode tab stack, e.g., an anode tab stack and/or a cathode tab stack. A first portion of the electrode tabs may be separated by one or more spacers between respective tabs. A second portion of each of the tabs adjacent to the first portion may extend beyond the spacer(s) and may be joined to the other electrode tabs in the stack, e.g., by bending the second portions of the tabs together adjacent to the first portion of the tabs that are separated by spacers. By joining the second portion of each tab together, a penetration weld may be formed to weld or otherwise attach the tabs to each other (e.g., in a manner that electrically couples the tabs to each other). The joined portion of the electrode tabs may be positioned adjacent to a conductive plate during the welding process to allow the penetration weld to also penetrate through the conductive plate and attach the joined tabs to the conductive plate. In the case of a medical device, the conductive plate may be electrically coupled to electronics of the medical device. In this manner, the conductive plate may be electrically coupled to the joined tabs to the electronics of the IMD. In some examples, one or more side welds may be formed along the side of the tabs at the first portion in which the tabs are separated by one or more spacers. In some examples, the joined portion of the tabs may be welded directly to the battery housing, e.g., where the weld penetrates through the joined portion of the tabs and partially or fully through the battery housing.

Examples of the disclosure may provide for one or more benefits. For example, a penetration weld may be stronger than a side weld along the side of a tabs stack in which the tabs are separated by spacer(s). Additionally, or alternatively, in an example configuration in which portions of each tabs are joined by a penetration weld, as described herein, there may be less stress concentration, more strain relief, and/or less residual stress, e.g., as compared to an electrode tab stack separated by spacers and attached only via one or more side welds on the side of the stack. Additionally, or alternatively, in an example configuration in which portions of each tabs are joined by a penetration weld, as described herein, the presence of the penetration weld may also reduce the mechanical load on the one or more side welds. The penetration weld and the side weld(s) may work together to reduce mechanical load on each weld.

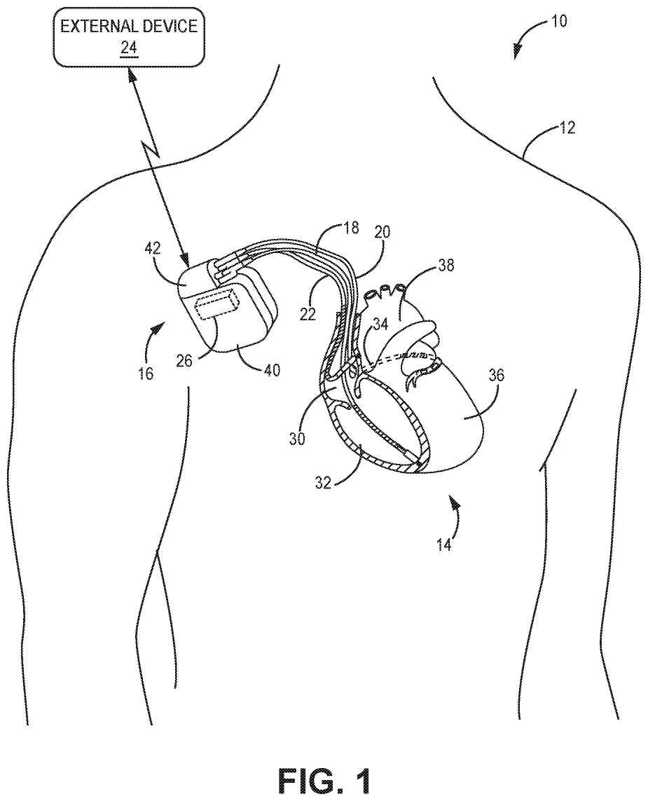

FIG. 1 is a conceptual diagram that illustrates an example medical device system 10 that may be used to provide electrical therapy to a patient 12. Patient 12 ordinarily, but not necessarily, will be a human. System 10 may include an IMD 16, and an external device 24. In the example illustrated in FIG. 1, IMD 16 has battery 26 positioned within an outer housing 40 of the IMD 16. Battery 26 may be a primary or secondary battery.

While the examples in the disclosure are primarily described with regard to battery 26 positioned within housing 40 of IMD 16 for delivery of electrical therapy to heart of patient 12, in other examples, battery 26 may be utilized with other implantable medical devices. For example, battery 26 may be utilized with an implantable drug delivery device, an implantable monitoring device that monitors one or more physiological parameter of patient 12, an implantable neurostimulator (e.g., a spinal cord stimulator, a deep brain stimulator, a pelvic floor stimulator, a peripheral nerve stimulator, or the like), or the like. Moreover, while examples of the disclosure are primarily described with regard to implantable medical devices, examples are not limited as such. Rather, some examples of the batteries described herein may be employed in any medical device including non-implantable medical devices. For example, an example battery may be employed to supply power to a medical device configured delivery therapy to a patient externally or via a transcutaneoulsy implanted lead or drug delivery catheter.

In the example depicted in FIG. 1, IMD 16 is connected (or “coupled”) to leads 18, 20, and 22. IMD 16 may be, for example, a device that provides cardiac rhythm management therapy to heart 14, and may include, for example, an implantable pacemaker, cardioverter, and/or defibrillator that provides therapy to heart 14 of patient 12 via electrodes coupled to one or more of leads 18, 20, and 22. In some examples, IMD 16 may deliver pacing pulses, but not cardioversion or defibrillation shocks, while in other examples, IMD 16 may deliver cardioversion or defibrillation shocks, but not pacing pulses. In addition, in further examples, IMD 16 may deliver pacing pulses, cardioversion shocks, and defibrillation shocks.

IMD 16 may include electronics and other internal components necessary or desirable for executing the functions associated with the device. In one example, IMD 16 includes one or more of processing circuitry, memory, a signal generation circuitry, sensing circuitry, telemetry circuitry, and a power source. In general, memory of IMD 16 may include computer-readable instructions that, when executed by a processor of the IMD, cause it to perform various functions attributed to the device herein. For example, processing circuitry of IMD 16 may control the signal generator and sensing circuitry according to instructions and/or data stored on memory to deliver therapy to patient 12 and perform other functions related to treating condition(s) of the patient with IMD 16.

IMD 16 may include or may be one or more processors or processing circuitry, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” and “processing circuitry” as used herein may refer to any of the foregoing structure or any other structure suitable for implementation of the techniques described herein.

Memory may include any volatile or non-volatile media, such as a random-access memory (RAM), read only memory (ROM), non-volatile RAM (NVRAM), electrically erasable programmable ROM (EEPROM), flash memory, and the like. Memory may be a storage device or other non-transitory medium.

The signal generation circuitry of IMD 16 may generate electrical therapy signals that are delivered to patient 12 via electrode(s) on one or more of leads 18, 20, and 22, in order to provide pacing signals or cardioversion/defibrillation shocks, as examples. The sensing circuitry of IMD 16 may monitor electrical signals from electrode(s) on leads 18, 20, and 22 of IMD 16 in order to monitor electrical activity of heart 14. In one example, the sensing circuitry may include switching circuitry to select which of the available electrodes on leads 18, 20, and 22 of IMD 16 are used to sense the heart activity. Additionally, the sensing circuitry of IMD 16 may include multiple detection channels, each of which includes an amplifier, as well as an analog-to-digital converter for digitizing the signal received from a sensing channel (e.g., electrogram signal processing by processing circuitry of the IMD).

Telemetry circuitry of IMD 16 may be used to communicate with another device, such as external device 24. Under the control of the processing circuitry of IMD 16, the telemetry circuitry may receive downlink telemetry from and send uplink telemetry to external device 24 with the aid of an antenna, which may be internal and/or external.

The various components of IMD 16 may be coupled to a power source such as battery 26, which may be a lithium primary battery. Battery 26 may be capable of holding a charge for several years. In general, battery 26 may supply power to one or more electrical components of IMD 16, such as, e.g., the signal generation circuitry, to allow IMD 16 to deliver therapy to patient 12, e.g., in the form of monitoring one or more patient parameters, delivery of electrical stimulation, or delivery of a therapeutic drug fluid. Battery 26 may include a lithium-containing anode and cathode including an active material that electrochemically reacts with the lithium within an electrolyte to generate power.

Leads 18, 20, 22 that are coupled to IMD 16 may extend into the heart 14 of patient 12 to sense electrical activity of heart 14 and/or deliver electrical therapy to heart 14. In the example shown in FIG. 1, right ventricular (RV) lead 18 extends through one or more veins (not shown), the superior vena cava (not shown), and right atrium 30, and into right ventricle 32. Left ventricular (LV) coronary sinus lead 20 extends through one or more veins, the vena cava, right atrium 30, and into the coronary sinus 34 to a region adjacent to the free wall of left ventricle 36 of heart 14. Right atrial (RA) lead 22 extends through one or more veins and the vena cava, and into the right atrium 30 of heart 14. In other examples, IMD 16 may deliver therapy to heart 14 from an extravascular tissue site in addition to or instead of delivering therapy via electrodes of intravascular leads 18, 20, 22. In the illustrated example, there are no electrodes located in left atrium 36. However, other examples may include electrodes in left atrium 36.

IMD 16 may sense electrical signals attendant to the depolarization and repolarization of heart 14 (e.g., cardiac signals) via electrodes (not shown in FIG. 1) coupled to at least one of the leads 18, 20, and 22. In some examples, IMD 16 provides pacing pulses to heart 14 based on the cardiac signals sensed within heart 14. The configurations of electrodes used by IMD 16 for sensing and pacing may be unipolar or bipolar. IMD 16 may also deliver defibrillation therapy and/or cardioversion therapy via electrodes located on at least one of the leads 18, 20, and 22. IMD 16 may detect arrhythmia of heart 14, such as fibrillation of ventricles 32 and 36, and deliver defibrillation therapy to heart 14 in the form of electrical shocks. In some examples, IMD 16 may be programmed to deliver a progression of therapies (e.g., shocks with increasing energy levels) until a fibrillation of heart 14 is stopped. IMD 16 may detect fibrillation by employing one or more fibrillation detection techniques known in the art. For example, IMD 16 may identify cardiac parameters of the cardiac signal (e.g., R-waves), and detect fibrillation based on the identified cardiac parameters.

In some examples, external device 24 may be a handheld computing device or a computer workstation. External device 24 may include a user interface that receives input from a user. The user interface may include, for example, a keypad and a display, which may be, for example, a cathode ray tube (CRT) display, a liquid crystal display (LCD) or light emitting diode (LED) display. The keypad may take the form of an alphanumeric keypad or a reduced set of keys associated with particular functions. External device 24 can additionally or alternatively include a peripheral pointing device, such as a mouse, via which a user may interact with the user interface. In some embodiments, a display of external device 24 may include a touch screen display, and a user may interact with external device 24 via the display.

A user, such as a physician, technician, other clinician or caregiver, or the patient, may interact with external device 24 to communicate with IMD 16. For example, the user may interact with external device 24 to retrieve physiological or diagnostic information from IMD 16. A user may also interact with external device 24 to program IMD 16 (e.g., select values for operational parameters of IMD 16).

External device 24 may communicate with IMD 16 via wireless communication using any techniques known in the art. Examples of communication techniques may include, for example, low frequency or radiofrequency (RF) telemetry, but other techniques are also contemplated. In some examples, external device 24 may include a communication head that may be placed proximate to the patient's body near the IMD 16 implant site in order to improve the quality or security of communication between IMD 16 and external device 24.

In the example depicted in FIG. 1, IMD 16 is connected (or “coupled”) to leads 18, 20, and 22. In the example, leads 18, 20, and 22 are connected to IMD 16 using the connector block 42. For example, leads 18, 20, and 22 are connected to IMD 16 using the lead connector ports in connector block 42. Once connected, leads 18, 20, and 22 are in electrical contact with the internal circuitry of IMD 16. Battery 26 may be positioned within the housing 40 of IMD 16. Housing 40 may be hermetically sealed and biologically inert. In some examples, housing 40 may be formed from a conductive material. For example, housing 40 may be formed from a material including, but not limited to, titanium, stainless steel, among others.

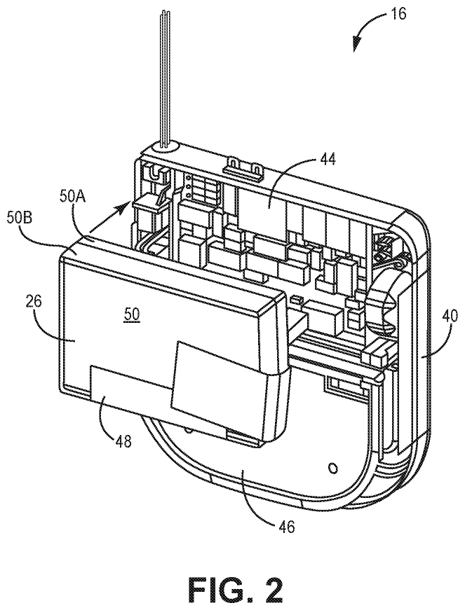

FIG. 2 is a conceptual diagram of IMD 16 of FIG. 1 with connector block 42 not shown and a portion of housing 40 removed to illustrate some of the internal components within housing 40. IMD 10 includes housing 40, a control circuitry 44 (which may include processing circuitry), battery 26 (e.g., an organic electrolyte battery) and capacitor(s) 46. Control circuitry 44 may be configured to control one or more sensing and/or therapy delivery processes from IMD 16 via leads 18, 20, and 22 (not shown in FIG. 2). Battery 26 includes battery assembly housing 50 and insulator 48 (or liner) disposed therearound. Battery 26 charges capacitor(s) 46 and powers control circuitry 44.

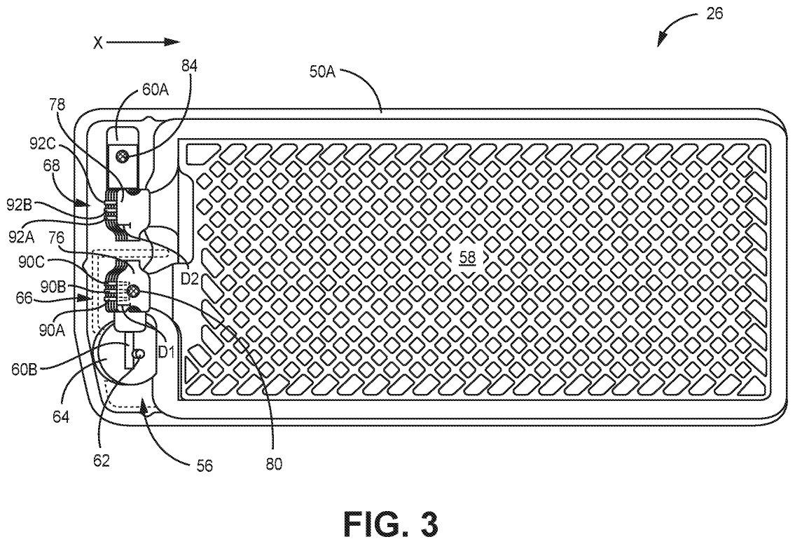

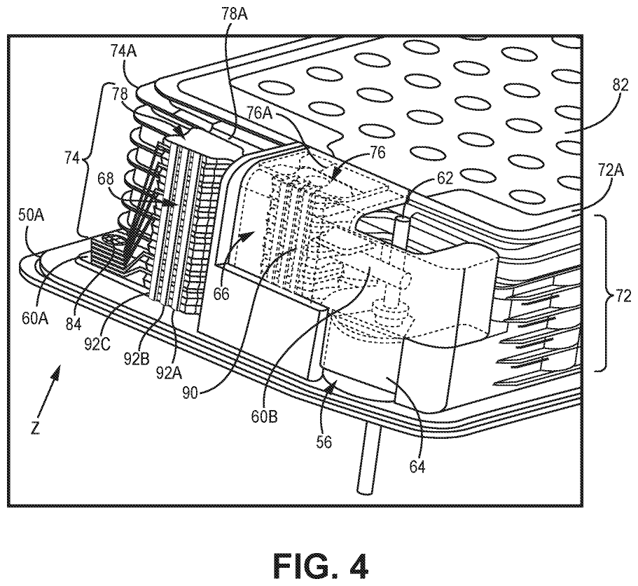

FIGS. 3 and 4 are conceptual diagrams illustrating aspects of example battery 26. Battery 26 includes assembly housing 50 having a bottom housing portion 50A and top housing portion 50B (shown in FIG. 2), a feed-through assembly 56, and an electrode assembly 58. An electrolyte may be filled into the enclosure via a fill port (not shown) in housing 50. Housing 50 houses electrode assembly 58 with the electrolyte. Top portion 50B and bottom portion 50A of housing may be welded or otherwise attached to seal the enclosed components of battery 26 within housing 50. Feed-through assembly 56, formed by pin 62 and insulator member/ferrule 64, is electrically connected to jumper pin 60B. The connection between pin 62 and jumper pin 60B to form the positive terminal of the battery. Conductor 60A is electrically connected to the housing 50A to form the negative terminal of the battery.

As noted above, a fill port (not shown) allows for the introduction of liquid electrolyte to electrode assembly 58. The electrolyte creates an ionic path between cathode 66 and anode 68 of electrode assembly 58. The electrolyte serves as a medium for migration of ions between cathode 66 and anode 68 during an electrochemical reaction with these electrodes.

Electrode assembly 58 is depicted as a stacked assembly. Cathode 66 comprises a set of electrode plates 72 (cathode electrode plates) with a set of tabs 76 extending therefrom in a stacked configuration. Although not shown in FIG. 3, one or more spacers may be located between respective tabs 76 of each plate 72. Side welds 90A-90C (collectively referred to as side welds 90) are located on the side of the set of tabs 76 and may penetrate into tabs 76 and spacers in approximately the X-direction (as labelled in FIG. 3). Side welds 90 may attach the respective individual tabs of set of tabs 76 to each other (e.g., in additions to the spacers that may be located between respective tabs in the stack).

Each electrode plate, such as plate 72A, includes a current collector or grid 82, a tab, such as tab 76A, extending therefrom, and an electrode material. Tabs 76 (e.g., tab 76A) and plates 72 may comprise a conductive material (e.g., aluminum, titanium, copper, and/or alloys thereof). Electrode material (or cathode material) may include metal oxides (e.g., vanadium oxide, silver vanadium oxide (SVO), manganese dioxide, etc.), carbon monofluoride and hybrids thereof (e.g., CFx+MnO2), combination silver vanadium oxide (CSVO), lithium ion, other rechargeable chemistries, or other suitable compounds.

Anode 68 may be constructed in a similar manner as cathode 66. Anode 68 includes a set of electrode plates 74 (anode electrode plates) with a set of tabs 78 extending therefrom in a stacked configuration. Although not shown in FIG. 3, one or more spacers may be located between respective tab 78 of each plate 74. Tabs 78 may be electrically coupled to conductive member 60A, which may be shaped as a plate, and may comprise titanium, nickel, niobium, tantalum, vanadiumor other suitable materials. Conductive member 60A allows anode 68 to be electrically coupled to electronic components outside of battery 26.

Side welds 92A-92C (collectively referred to as side welds 92) are located on the side of the set of tabs 78 and penetrate into tabs 78 in approximately the X-direction (as labelled in FIG. 3). Side welds 92 may attach the respective individual tabs of set of tabs 78 to each other (e.g., in addition to one or more spacers that may be located between respective tabs in the stack). In addition to, or as an alternative to, one or more side welds (such as side welds 92A-92C to attach the stack of tabs 78 and spacers to each other, a rivet or other alignment member may be employed that extends through an aperture formed through the stack of tabs and spacers to attach the tabs 78 and spacers to each other, e.g., by mechanical fastening.

In accordance with some examples of the disclosure, a portion of each of tabs 78 that extends beyond the area of the stack including spacer(s) may be joined to each other, e.g., by bending or otherwise drawing the tabs together adjacent to the portion of the stack including spacer(s). At the portion of the tabs that are joined together, penetration weld 84 may be formed that penetrates through each of tabs 78 to attach the individual tabs 78 to each other. The joined portion of tabs 78 may be located adjacent to a surface of conductive member 60A such that penetration weld 84 extends into or through conductive member 60A to attach the joined tabs 78 to conductive member 60A. In such a configuration, tabs 78 may be electrically coupled to each other and to conductive member 60A. It may be preferable to have penetration weld 84 melt through or otherwise extend all the way through conductive member 60A to allow for visual inspection of the melt spot at the bottom of conductive member 60A. The presence of the melt spot of weld 84 indicates fusion is achieved between all tabs 78A-78E and conductive member 60A. In other examples, weld 84 may only extend partially through conductive member 60A.

While the example of FIGS. 3 and 4 show a single penetration weld 84, in other examples, multiple penetration welds may be formed to attach tabs 78 to each other and/or conductive member 60A in the manner described herein.

Each anode electrode plate, such as plate 74A, includes a current collector (not shown) or grid, an electrode material and a tab, such as tab 78A, extending therefrom. Tabs 78 and plates 74 may comprise a conductive material (e.g., aluminum, titanium, copper, nickel, and/or alloys thereof). The electrode material (or anode material) may include elements from Group IA, IIA or IIIB of the periodic table of elements (e.g. lithium, sodium, potassium, etc.), alloys thereof, intermetallic compounds (e.g. Li—Si, Li—B, Li—Si—B etc.), or an alkali metal (e.g. lithium, etc.) in metallic form.

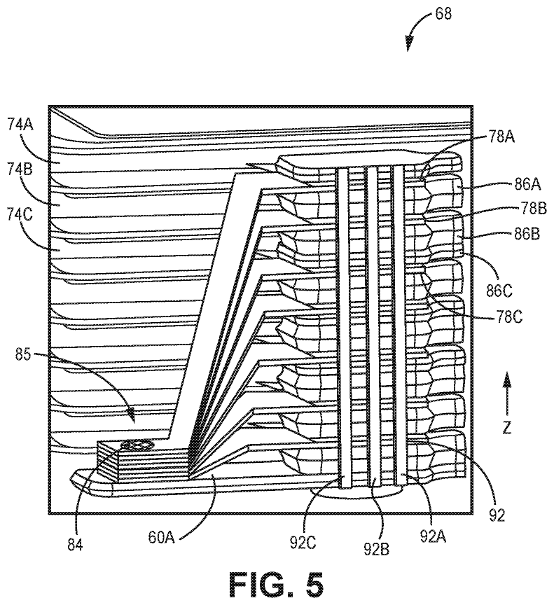

FIG. 5 is a conceptual schematic diagram illustrating a magnified view of a portion of anode 68 of battery 26. As shown, electrodes plates 74 of anode 68 includes anode electrode plates 74A, 74B, 74C (among others) in a stacked configuration. Anode tabs 78A, 78B, 78C extend from anode electrodes plates 74A, 74B, 74C, respectively, and exhibit the same stacked configuration as electrode plates 74. At least one spacer is located between each respective tab 78. For example, spacer 86A is located between tabs 78A and tab 78B, and two spacers 86B and 86C are located between tab 78B and tab 78C.

For ease of description and illustration, not all the tabs and spacers of anode 68 are labelled in FIG. 5. However, it is understood that the description of tabs 78A-78C and spacers 86A-86C also may apply to any of the tabs and spacers shown in FIG. 5. Additionally, while FIG. 5 is described with regard to anode 68 it is contemplated that the same configuration is applicable to cathode 66 of battery 26 shown in FIG. 3.

In some examples, spacer 86A ensures tabs 78A and 78B are substantially straight extending from plates 74A and 74B, respectively, and are not bent during a subassembly process to stack the set of tabs 78 and plates 74 for anode 68. While a single spacer 86A is depicted as being placed between two tabs, more than one spacer may be placed between two tabs, such as, e.g., spacers 86B and 86C between tabs 78B and 78C.

Spacers 86A-86C may comprise an electrically conductive material, e.g., such that the each of the tabs 78 are electrically interconnected at least in part via spacers 86. Example materials for spacers 86 may include titanium. nickel, alloys thereof or other suitable materials. In other examples, spacers 86 may be an electrically insulating material, e.g., such that spacers 86 do not electrically couple the individual tabs 78 to each other. In either instance, tabs 78 may be electrically coupled to each other by joining portions 85 of each tab 78 together (e.g., directly in contact with each other without being separated by spacers) and forming a penetration weld 84 to attach the individual tabs 78 to each other. Penetration weld 84 may also attach and electrically couple tabs 78 to conductive member 60A, as shown in FIG. 5. In some examples, a rivet or other attachment member may be employed that extends through an aperture formed in the stack of spacers 86 and tabs 78 to mechanically fasten the stack of tabs 78 and spacers 86 to each other, e.g., in instances in which spacers 86 are electrically insulating material and suitable side welds 92 may be difficult to form.

While portion 85 of tabs 78 are joined to conductive member 60A in the illustrated example of FIG. 4, in some examples, the joined portion 85 of tabs 78 may be similarly attached to housing 50A, e.g., without conductive plate 60A interposed between, by forming penetration weld 84 through tabs 78 and partially or full through housing 50A.

Spacers 86A-86C may include a variety of shapes. Exemplary spacers include a substantially H-shaped spacer, substantially rectangular, circular, or include at least one triangular shape (e.g. a single triangle, a hexagon etc.). Spacers 86A-86C may have different or substantially the same individual thicknesses in the z-direction labeled in FIG. 5, e.g., to achieve different design criteria. For example, a thicker electrode plate may requires a thicker spacer. In the example in of FIG. 5, spacer 86A may have substantially the same thickness of spacer 86B but spacer 86C may be thinner than spacers 86A and 86B. Examples of spacers 86A-86C may include one or more of the example spacers described in U.S. Published Patent Application No. 2009/0197180.

As noted above, side welds 92 may penetrate into tabs 78. As shown in FIG. 5, side welds 92 also penetrate into spacers 86 (including spacers 86A-86C). In such examples, spacers 86 may formed of material suitable for being welded to each other, tabs 78 and/or alignment member 84. Example materials for spacers 86 may include titanium and the like.

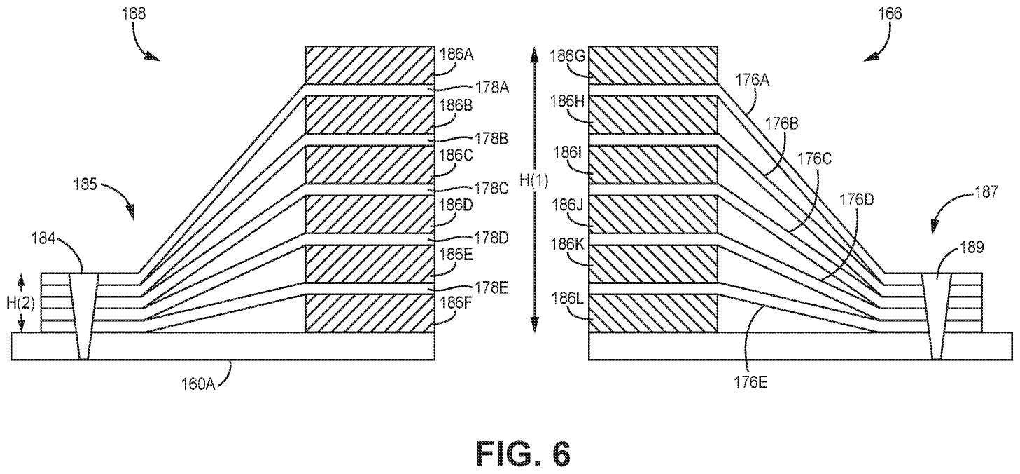

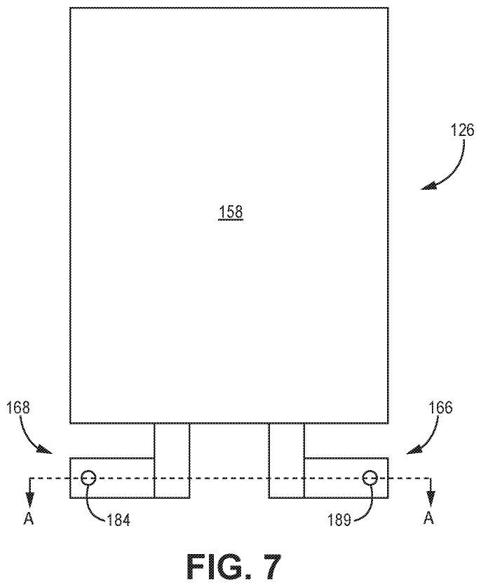

FIG. 7 is a conceptual diagram illustrating a plan view of an example battery 126 including electrode assembly 158 which includes anode 168 and cathode 166. Battery 126 may be substantially similar to that of battery 26 described previously and shown, e.g., in FIGS. 3-5. As such, similar features are similarly numbered (e.g., anode 68 is substantially similar to anode 168, electrode assembly 58 is substantially similar to assembly 158, and so forth). FIG. 6 is a simplified cross-section view of an example portion of anode 168 and cathode 166 along cross-section A-A shown in FIG. 7.

As shown in FIG. 6, anode 168 includes a plurality of tabs 178A-178E and spacers 186A-186F. Anode 168 may be substantially similar to that of anode 68 described previously and shown in FIGS. 3-5. Tabs 178A-178E extend from a current collector or grid of an anode plate (not labelled in FIGS. 6 and 7) of electrode assembly 58. However, unlike that of anode 68, anode 168 includes five individual tabs 178A-178E rather than the seven tabs shown for anode 68. Anode 168 also includes spacers 186A-186F, with spacer 186A being located on top of tab 178A, spacers 186B-186E being between a first portion of respective tabs 178A-178E (e.g., spacer 186B is between a portion of tab 178A and a portion of tab 178B), and spacer 86F being between tab 78E and conductive member 160A. As such, in the stacked configuration shown in FIG. 6, a first portion of tabs 178A-178E are separated from each other by spacers 186B-186E.

Tabs 178A-178E and spacers 186B-186E may have any suitable composition and thickness (in the H(1) direction shown in FIG. 6). In some examples, tabs 178A-178E and spacers 186B-186E are both electrically conductive (e.g., where tabs 178A-178E and spacers 186B-186E have substantially the same composition), while in other examples, tabs 178A-178E are electrically conductive and spacers 186B-186E are electrically insulative. In some examples, tabs 178A-178E may comprise copper, aluminum, titanium (e.g., pure titanium), or alloys thereof. In some examples, spacers 186B-186E may comprise copper, aluminum, titanium (e.g., pure titanium), nickel, or alloys thereof. In some examples, when electrically insulative, spacers 186B-186E may comprise polymeric materials such as polypropylene, polyethylene and ethylene tetrafluoroethylene (ETFE). In some examples, each of tabs 178A-178E may have a thickness of about 0.001 inch to about 0.006 inch, such as, about 0.003 inch to about 0.005 inch. The thickness of each of individual tab may be substantially uniform or nonuniform, and may be the same or different from other tabs of tabs 178A-178E. In some examples, each of spacers 186B-186E may have a thickness of about 0.005 inch to about 0.040 inch, such as, about 0.010 inch to about 0.020 inch. The thickness of each individual spacers may be substantially uniform or nonuniform, and may be the same or different from other spacers of spacers 186B-186E. Other values are contemplated.

As shown, another portion 185 of each of tabs 178A-178E extends beyond the location at which tabs 178A-178E are stacked with spacers 186B-186E. At that portion 185, tabs 178A-178E are joined together with each other. For example, as shown in FIG. 6, each of spacers 186B-186E are bent to some degree out of the plane in which the opposing portion of spacers 186B-186E are stacked with spacers 186B-186E. In this manner, portion 185 of each of tabs 178A-178E may be brought into closer proximity to each other, e.g., directly in contact with each other without any spacers position in between. In the example of FIG. 6, portions 185 of each of tabs 178A-178E are joined adjacent to conductive member 60A, which is located on the bottom of the stack of spacers 186B-186E and tabs 178A-178E. Thus, the top tab 178A is bent more than the bottom tab 178E since the joining location of tabs 178A-178E at portion 185 is further from the plane of tab 178A between spacers 186A and 186B compared to tab 178E between spacers 186E and 186F. While tabs 178A-178E are shown being joined near the bottom most tab, in other examples, tabs 178A-178E may be bent or otherwise formed to be joined at a location nearest a middle tab in the stack of tabs or near the top most tab in the stack of tabs.

As shown in FIG. 6, in the location where portion 185 of each of tabs 178A-178E are joined, penetration weld 184 may be formed to attach each of tabs 178A-178E. When joined with each other, tabs 178A-178E may be electrically coupled to each other. Additionally, penetration weld 184 may also extends into or through conductive member 160A to attached portion 185 of tabs 178A-178E to conductive member 160A. In such a configuration, tabs 178A-178E may be electrically coupled to conductive member 160A. It may be preferable to have penetration weld 184 melt through or otherwise extend all the way through conductive member 160A to allow for visual inspection of the melt spot at the bottom of conductive member 160A. The presence of the melt spot of weld 184 indicates fusion is achieved between all tabs 178A-178E and conductive member 160A. In other examples, weld 184 may only extend partially through conductive member 160A.

As shown in FIG. 6, the stack of tabs 178A-178E and spacers 186B-186E may define a total height of H(1). Conversely, portion 185 where tabs 178A-178E are joined together may define a height of H(2). In some examples, the reduced height of portion 185 and/or lack of spacers at portion 185 may allow for a penetration weld through each of tabs 178A-178E, e.g., where it may not otherwise be feasible to form such a penetration weld through the stack of tabs 178A-178E and spacers 186B-186E. Height H(1) may be greater than height H(2), e.g., as a result of the thickness of spacers 186B-186E. In some examples, height H(2) may be less than 25% of height H(1), such as about 10% to about 25% of height H(1). In some examples, height H(2) may be about 0.05 inch to about 0.25 inch, such as, about 0.20 inch to about 0.23 inch. In some examples, height H(1) may be about 0.005 inch to about 0.06 inch, such as, about 0.024 inch to about 0.045 inch. Other values are contemplated.

As shown in the example of FIG. 6, cathode 166 may have substantially the same configuration of tabs 176A-176E and spacers 186G-186L as that described for anode 168. A portion of tabs 176A-176E may be stacked with spacers 186G-186L, while another portion 187 of tabs 176A-176E may be joined together with other. Penetration weld 189 may be formed to attach tabs 176A-176E to each other and to conductive member 60B.

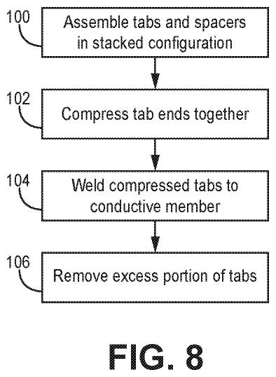

FIG. 8 is an example flow diagram illustrating an example technique for assembling a battery, such as battery 26 or battery 126. For ease of description, the example technique will be described with regards to battery 126.

As shown in the example of FIG. 6, tabs 176A-176E and 178A-178E, and spacers 186A-186L may be assembled such that a portion of the tabs 176A-176E and tabs 178A-178E are separated by spacers 186G-186L in a stacked configuration (100). For example, the individual electrode plates of electrode assembly 158 and corresponding spacers (e.g., spacers 186A-186L) may be sequentially stacked onto each other by placing a cathode plate for one of tabs 176A-176E in a fixture, followed by placement of an anode plate for one of tab 178A-178E on top of that cathode plate, as so forth, along with one or more spacers between the tabs to arrive at an assembly having a cross-section similar to that of FIG. 6.

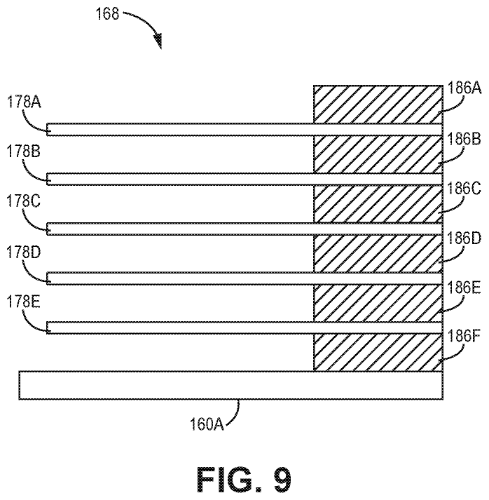

However, if tabs 176A-176E and 178A-178E are initially straight (e.g., substantially planar) during the assembly process, portions 185 and 187 may not be joined with each other after the plates and spacers have been assembled in a stacked configuration as described above. For example, FIG. 9 is a conceptual diagram illustrating anode 168 following the initial assembly to stack the plates and spacers, but prior to joining tabs 178A-178E at portion 185. As shown, each of 178A-178E are initially straight following the assembly of the electrode plates and spacers in a stacked configuration.

As such, the ends of tabs 178A-178E not separated by spacers 186A-186F may need to be compressed to bend or otherwise deformed to join portions 185 of tabs 178A-178E together (102) to achieve the configuration shown in FIG. 6. For example, the “free” end of tabs 178A-178E may be bent together by pushing the tabs from 178A towards 178E using a tool with a flat tip. In some examples, when joined together, portion 185 of tabs 178A-178E may be directly adjacent to each other (e.g., in direct contact).

Once the “free” ends at portion 185 of tabs 178A-178E have been compressed and held together, e.g., penetration weld 184 may be formed in portion 185 of tabs 178A-178E (106). Penetration weld 185 may extend through each of tabs 178A-178E to attach the individual tabs to each other. Any suitable technique may be employed to form penetration weld 185 including, e.g., laser welding or electron beam welding. For example, an energy source such as a laser may be directed at the top surface of tab 178A which then melts the material through the entire stack of joined tabs 178A-178E. In some examples, the joined tabs 178A-178E may be positioned over a surface of conductive member 160A during the welding process so that penetration weld 184 extends partially into or through conductive plate 160A.

One or more side welds along the side of tabs 178A-178E in the area of spacers 186A-186F, such as side welds 92 shown in FIG. 3-5, may be formed before, during, or after penetration weld 184 is formed. In some examples, the side weld(s) 92 may be formed prior to the penetration weld but after assembling the plates and spacers in a stacked configuration, e.g., to prevent relative movement between tabs 178A-178E during the process to bend or otherwise join tabs 178A-178E with each other followed by the formation of penetration weld 184.

In some examples, once penetration weld 184 has been formed, a portion of the ends of one or more tabs 178A-178E may be trimmed or otherwise removed (104). For example, in cases in which all the tabs are the same length initially when in the planar or straight configuration, some tabs may be bent more than other to join the tabs together. As such, the edges of the joined ends of tabs 178A-178E may not by aligned, e.g., the end of bottom tab 178F may extend beyond the end of top tab 178A since top tab 178A was required to be bent further when joining portion 185 of tabs 178A-178E together. As such, a portion of the ends of one or more of tabs 178A-178E may be trimmed, e.g., so that all the tab ends terminate at the same position. In some example, tabs 178A-178E may be trimmed by, e.g., a cutting tool such as diagonal pliers. In other examples, the length of each tabs 178A-178E may be initially provided in the straight or planar configuration so that the free ends are aligned with each other after tabs 178A-178E are bent, e.g., in the example of FIG. 6, the bottom tab 178F may be the shortest in length in the stack of tabs 178A-178E prior to bending while the top tab 178A may be the longest in length the example of FIG. 6.

Various examples have been described in the disclosure. These and other examples are within the scope of the following claims.

1. A battery assembly for an implantable medical device, the assembly comprising:
an electrode stack comprising a plurality of electrode plates, wherein the plurality of electrode plates comprises a first electrode plate including a first tab extending from the first electrode plate and a second electrode plate including a second tab extending from the second electrode plate;
a spacer between a first portion of the first tab and a second portion of the second tab, wherein a third portion of the first tab and a fourth portion of the second tab are joined together adjacent to the first portion, second portion, and the spacer; and
a penetration weld that extends through the third portion of the first tab and the fourth portion of the second tab.

2. The assembly of claim 1, wherein the first portion, the second portion, and the spacer define a first height, and wherein the third portion of the first tab and the fourth portion of the second tab joined together define a second height less than the first height.

3. The assembly of claim 1, wherein the third portion of the first tab and the fourth portion of the second tab are in direct contact with each other at an interface.

4. The assembly of claim 3, wherein the penetration weld extends through at least a portion of the interface.

5. The assembly of claim 1, further comprising an electrically conductive member, wherein the third portion of the first tab and the fourth portion of the second tab are connected to the electrically conductive member via the penetration weld, wherein the electrically conductive member is configured to electrically couple the first tab and the second tab to electronics of a medical device.

6. The assembly of claim 1, wherein the first tab and the second tab are formed of at least one of copper, aluminum, titanium, nickel, or alloys thereof.

7. The assembly of claim 1, further comprising a weld on a side of the electrode stack extending from the first tab to the second tab across the spacer.

8. The assembly of claim 1, wherein the first electrode plate comprises a first anode electrode plate and the second electrode plate comprises a second anode electrode plate.

9. The assembly of claim 1, wherein the penetration weld comprises a laser penetration weld.

10. The assembly of claim 1, further comprising a rivet that extends through the first portion of the first tab, the spacer, and the second portion of the second tab to mechanically fasten the first tab, the spacer, and the second tab to each other.

11. A method for forming a battery assembly, the method comprising:
assembling an electrode stack with a spacer, electrode stack comprising a plurality of electrode plates, wherein the plurality of electrode plates comprises a first electrode plate including a first tab extending from the first electrode plate and a second electrode plate including a second tab extending from the second electrode plate, wherein the spacer is between a first portion of the first tab and a second portion of the second tab when the electrode stack is assembled with the spacer;
joining a third portion of the first tab and a fourth portion of the second tab together adjacent to the first portion, second portion, and the spacer; and
welding the electrode stack to form a penetration weld that extends through the third portion of the first tab and the fourth portion of the second tab.

12. The method of claim 11, wherein joining the third portion of the first tab and the fourth portion of the second tab together comprising bending at least one of the first tab or the second tab.

13. The method of claim 11, further comprising trimming a free end of at least one of the first tab or the second tab adjacent to the penetration weld.

14. The method of claim 11, wherein the first portion, the second portion, and the spacer define a first height, and wherein the third portion of the first tab and the fourth portion of the second tab joined together define a second height less than the first height.

15. The method of claim 11, wherein the third portion of the first tab and the fourth portion of the second tab are in direct contact with each other at an interface.

16. The method of claim 15, wherein the penetration weld extends through at least a portion of the interface.

17. The method of claim 11, further comprising an electrically conductive member, wherein the third portion of the first tab and the fourth portion of the second tab are connected to the electrically conductive member via the penetration weld, wherein the electrically conductive member is configured to electrically couple the first tab and the second tab to electronics of a medical device.

18. The method of claim 11, wherein the first tab and the second tab are formed of at least one of copper, aluminum, titanium, nickel, or alloys thereof.

19. The method of claim 11, further comprising forming a weld on a side of the electrode stack extending from the first tab to the second tab across the spacer.

20. The method of claim 11, wherein the first electrode plate comprises a first anode electrode plate and the second electrode plate comprises a second anode electrode plate.

21. The method of claim 11, wherein welding the electrode stack to form the penetration weld comprises laser welding the electrode stack to form the penetration weld.

22. An implantable medical device comprising:
an outer housing;
processing circuitry; and
the battery assembly of claim 1 within the outer housing, wherein the processing circuitry is configured to control delivery of electrical therapy from the implantable medical device to a patient using power supplied by the battery assembly.
